(19) United States
(12) Patent Application Publication
(10) Pub. No.: US 2024/0123119 A1
(43) Pub. Date: Apr. 18, 2024

(54) Microfiber Implant Made by Winding Filament

(71) Applicant: Asante Bio LLC, Norfolk, VA (US)

(72) Inventors: Michael Francis, Norfolk, VA (US); Heather Amin, Odessa, FL (US)

(73) Assignee: Asante Bio LLC, Norfolk, VA (US)

(21) Appl. No.: 18/485,387

(22) Filed: Oct. 12, 2023

Related U.S. Application Data
(60) Provisional application No. 63/379,714, filed on Oct. 14, 2022.

(51) Int. Cl. A61L 27/34; A61L 27/24; D04H 3/011; D04H 3/016; D04H 3/073; D04H 3/12; D04H 3/14; D06M 15/15

(52) U.S. Cl. CPC A61L 27/34; A61L 27/24; D04H 3/011; D04H 3/016; D04H 3/073; D04H 3/12; D04H 3/14; D06M 15/15; D06M 2101/32; D10B 2401/063; D10B 2509/00

(57) ABSTRACT

A method of making a microfiber implant. The method uses a fabrication apparatus that comprises a winding platform and a feeder head. The method comprises advancing a microfilament out of the feeder head towards the winding platform and making repeated windings of microfilament around the winding platform. While making the windings, the feeder head moves laterally relative to the winding platform. The feeder head could make multiple sweeps to stack layers of windings. This results in a microfiber patch that can then undergo further processing (e.g. applying a collagen coating) to result in the microfiber implant. Also disclosed are microfiber implants made by this winding technique and apparatus for performing the windings.

### TECHNICAL FIELD

This invention relates to making of microfibrous surgical implants for soft tissue repair, such as rotator cuff tendon repair.

### BACKGROUND

Manufacturing microfibrous tissue-engineered surgical implants by additive manufacturing techniques (such as 3D printing) are currently being investigated. Particularly in orthopedic medicine, research on collagen-containing microfibrous implants has advanced sufficiently to mimic the aligned collagen fibers of native ligaments and tendons. These aligned collagen fibers provide a scaffold for promoting ligament and tendon healing. There are many techniques for making microfibrous implants, such as electrospinning, wet extrusion, dry spinning, fused fiber fabrication (FFF) 3D printing, and conventional biotextile approaches (e.g. braiding, knitting, and weaving).

However, such existing techniques have various serious disadvantages. Electrospinning is limited by scaling and manufacturing complications, such as high batch variability, poor in-vivo cellular infiltration due to the limited porosity, and the use of harmful processing solvents. Although FFF printing has been explored for making ligament and tendon analogs, this technique is inherently limited to simple fused structures that cannot resemble native tissue structures and their strength. Biotextile techniques are overly complicated and expensive for mass production. Thus, alternative techniques for making microfibrous implants are needed.

### SUMMARY

This invention relates to a microfiber implant made by winding a microfilament on a fabrication apparatus. The microfiber implant could be used in surgically repairing various types of fibrous connective tissue (e.g. tendons, fascia, ligament, muscle, dermis, etc.) of musculoskeletal tissue (e.g. muscles or bone). Examples of various types of fibrous connective tissue that could be treated include the rotator cuff tendons, patellar tendon, Achilles tendon, pelvic or abdominal fascia, anterior cruciate ligament, skin, dura, etc. One particular setting in which the microfiber implant could be used is rotator cuff repair. Other clinical settings in which the implant could be used are explained below.

As used herein, different terms are used to differentiate the various stages of the product being fabricated, i.e. microfiber patch versus microfiber implant. The term “microfiber patch” means an intermediate product made by winding the microfilament on the fabrication apparatus. After fabrication is finished on the apparatus, the microfiber patch may need to undergo further processing to become the final desired product. The term “microfiber implant” means the final product that has undergone any necessary post-processing of the microfiber patch. Examples of such post-processing are described below.

FABRICATION APPARATUS. In one aspect, this invention is a fabrication apparatus for making a microfiber patch. The apparatus comprises one or more winding platforms upon which a microfiber patch is fabricated by winding of one or more microfilaments. Microfilament. The microfilament comprises a polymer material. Any suitable polymer material could be used, including biologic or synthetic polymers. Examples of suitable polymers include polydioxanone (PDO); poly(lactic-co-glycolic acid) (PLGA); poly(L-lactide) (PLLA); polyether ether ketone (PEEK); polycaprolactone (PCL); ultra-high molecular weight polyethylene (UHMWPE); collagen; carbon fiber; or nanocellulose. The microfilament could comprise a mixture of different polymers in any suitable ratio. The microfilament is very thin (micron-sized diameter). For example, the microfilament could have a diameter in the range of 5-125 μm. The microfilament could comprise a single filament (monofilament) or multiple filaments (multifilament or yarn).

Winding Platform. The winding platform could be any structure around which the microfilament could be wound while the platform is rotating. Examples of winding platforms include oval drum mandrel, cylindrical mandrel, or flat plate mandrel. The winding platform could have a regular or irregular shape. The winding platform could have any suitable cross-sectional size (e.g. diameter) depending on factors such as the size of the microfiber patch. For example, the diameter (or longest cross-sectional width) of the winding platform could be in the range of 0.1-100 cm.

The winding platform could have a non-stick coating (e.g. Teflon, polytetrafluoro-ethylene/PTFE) or be anodized to facilitate removal of the microfiber patch that is wound thereon. The winding platform could be tapered (made narrower) at one or both ends to facilitate microfiber patch removal. The winding platform could be dynamically sized. For example, it may be made to shrink in diameter to facilitate removal of the microfiber patch.

The apparatus could further comprise one or more brackets onto which the winding platform is mounted. For example, the apparatus could have two brackets that hold each end of the winding platform. The winding platform could be detachable from the bracket to facilitate removal of the microfiber patch. The fabrication apparatus is designed such that the winding platform rotates around an axis of rotation. This axis of rotation could be aligned in any suitable direction relative to the winding platform. The apparatus could further comprise one or more turn shafts for rotating the winding platforms.

Loom Frame. In some embodiments, the winding platform comprises crossbars of a loom frame. That is, the apparatus comprises a loom frame, which comprises two or more crossbars (i.e. the winding platform) upon which the microfilament is wound. The crossbars may be aligned at any suitable angle relative to each other depending on various factors such as the desired shape of the microfiber implant. For example, the crossbars could be aligned substantially parallel to each other. The crossbars could have any suitable shape on cross-section, such as round (cylindrical), oval, square, flat, or polygonal. The crossbars could have any suitable cross-sectional size (e.g. diameter) depending on factors such as the size of the microfiber patch. For example, the diameter (or longest cross-sectional width) of each crossbar could be in the range of 0.1-100 cm. The crossbars could have any suitable length depending on factors such as the size of the desired microfiber patch. For example, the length of each crossbar is 2.0-90 cm long. The axis of rotation for the loom frame could be substantially parallel to the crossbars.

Feeder Head. The fabrication apparatus further comprises one or more feeder heads for feeding the microfilament onto the winding platform. The microfilament is fed into the feeder head and then passed out of the feeder head. The microfilament is passed out of the feeder head and advanced towards the winding platform as it is rotating. Feeding of the microfilament into and out of the feeder head could be performed by passive or active means. One simple passive feeding mechanism is by rotational traction on the microfilament as the winding platform turns (i.e. the winding platform “pulls in” the microfilament). Further explanation about the winding of the microfilament by this mechanism is given below.

In performing windings, the feeder head moves laterally (translational motion) relative to the winding platform. The translational motion of the feeder head could vary according to the particular design of the microfiber patch, such as variations in direction, continuity (continuous or intermittent), speed, pauses, etc. The feeder head could have an adjustable angle to vary the directional angle for passing out the microfilament towards the winding platform. The adjustable angle could be dynamic during the fabrication process. The adjustable angle could be in one or multiple (two or more) axes. Also, the feeder head could be configured for multiaxial motion (two or three axes) relative to the winding platform to guide the microfilament towards the desired configuration. For example, the feeder head could move laterally relative to the winding platform and also move towards/away from the winding platform.

The feeder head could comprise a coating bath through which the microfilament travels before exiting out. In an alternate apparatus design, the coating bath could be located external to the feeder head. For example, the microfilament could travel through the external coating bath after exiting the feeder head. The coating bath contains a coating material for coating the microfilament. This coating could serve a variety of beneficial purposes such as a lubricant for the windings or enhance the therapeutic efficacy of the microfiber implant. Examples of possible coating materials include biologic materials (such as collagen or other components of extracellular matrix, cells, growth factors, etc), pharmaceutical agents such as small molecule drugs, bone-mimetic materials such as calcium sodium phosphosilicate (e.g. “Bioglass”) or other surface reactive glass-ceramic biomaterials, surfactants such as “Pluronic” poloxamers, solvents such as organic solvents or aqueous solutions (such as buffers or plain water), or materials that facilitate binding or joining of the microfilaments together (such as resins).

Heating. The fabrication apparatus could be designed such that the winding platform can be heated. The purpose of this heating is explained below. Heat may be generated in any suitable manner. For example, the winding platform could be made of an electrically conductive metal and an electric current is passed therethrough. Heat from the induced electrical resistance causes heating of the winding platform. In another example, a heating element could be contained inside the winding platform. Alternatively or in addition thereto, the fabrication apparatus could have a separate heat source for applying heat to the microfiber patch, such as a laser or infrared heater.

Additional Features. The fabrication apparatus may further comprise one or more filament holders for holding the supply of microfilament that is being fed into the feeder head. Examples of filament holders include spool, spinning reel, circular tray, spindle, roller, etc. If the feeder head has a coating bath (as described above), the apparatus could also further comprise a reservoir for holding and supplying the coating material. The reservoir is connected to the feeder head (e.g. a connection tube traveling from the reservoir to the coating bath).

FABRICATION METHOD. In another aspect, this invention is a method of making a microfiber implant. The method may use a fabrication apparatus as described above. The microfiber patch (precursor or intermediate to the microfiber implant) is made by a winding process on the fabrication apparatus. The microfilament is wound multiple times around the winding platform of the fabrication apparatus.

In the winding process, the winding platform spins about its rotation axis. The microfilament is fed into the feeder head. The microfilament is passed out of the feeder head and advanced towards the winding platform as it is rotating. The output rate of microfilament from the feeder head could be in the range of 25-800 cm/min. The microfilament is captured on the winding platform. The microfilament continues to be fed into and out of the feeder head in conjunction with lateral translational motion of the feeder head. The lateral (translation) travel speed of the feeder head could be in the range of 20-500 mm/min. The preceding steps are performed repeatedly such that the microfilament winds around the winding platform.

For a loom frame, a single winding on the crossbars means that the microfilament travels around the first crossbar, across to the second crossbar, around the second crossbar, (and around and across any additional crossbars), and back across to the first crossbar. This loop constitutes a single winding of the microfilament. Multiple such windings are performed to make the microfiber patch.

Each winding could be placed adjacent to the previous winding. The adjacent windings do not necessarily have to be in touch contact with each other. For example, having a small gap between the windings may be useful for creating pores or grooves that facilitate the integration (mechanical or biological) of the microfiber implant with the surrounding tissue. Multiple microfilaments (two or more; for example, up to 10) may be deposited simultaneously on the winding platform.

Multiple sweeps (two or more) of microfilament windings could be performed. This could stack sets of windings on top of each other. Each sweep of windings on the winding platform could make a single matting layer for the microfiber patch. As such, the microfiber patch could be constructed of multiple matting layers stacked on top of each other. The alternating sweeps may be in any direction such as unilateral (e.g. reset back to initial position and forward direction only), bidirectional (e.g. back and forth in both forward/reverse directions), or combinations thereof.

For example, a first sweep could make a first matting layer, a second sweep could make a second matting layer on top of the first matting layer, a third sweep could make a third matting layer on top of the second matting layer, and so on. With multiple sweeps, this process could make a microfiber patch with multiple matting layers. The number of sweeps across the winding platform could be in the range of 3-50. Each sweep could form a matting layer. This could make a microfiber patch having 3-50 matting layers. Each sweep across the winding platform could make 3-70 windings of the microfilament per centimeter across the winding platform.

The repeated winding process could be performed in varying degrees of continuity such as continuously, intermittently, with interruptions, etc. The microfiber patch could be made from a single continuous unbroken microfilament from beginning to end. Alternatively, there may be breaks in the microfilament. That is, the microfiber patch could be made from multiple (two or more) separate strands of microfilament. For example, there may be a break in the microfilament at the end of each sweep, and each matting layer is made from a separate strand of the microfilament.

In embodiments where the feeder head comprises a coating bath, the microfilament travels therethrough and becomes coated. The microfiber patch could be made from more than one type of microfilament. For example, multiple microfilaments of different sizes or material compositions could be combined. For example, one type of microfilament could be used to make one matting layer, and then a different type of microfilament used to make the next matting layer.

Heating or Fiber Fusion. The fabrication method could further comprise heating the winding platform or a part thereof. For a loom frame, one or more crossbars of the loom frame could be heated. This heating may be performed during the microfilament winding or after the windings are completed. Portions of the windings that are in contact with the heated parts of the winding platform (e.g. crossbar) would undergo melting or softening such that the microfilaments become heat bonded. This creates one or more fused regions on the microfiber patch. These fused regions could serve as borders or regions of stability in the microfiber implant. Alternatively, in situations where a binder (e.g. collagen coating) is applied to the microfilament(s) or microfiber patch, heating may cause the binder to meld therewith (e.g. by polymerizing, hardening, transitioning from liquid to gel/solid, etc). This melding of the binder strengthens the resulting microfiber implant.

There are many possible variations of this heating process. For example, the individual crossbars of the loom frame could be heated to different temperatures. In another example, only certain sections of the winding platform could be heated. Alternatively or in addition to heating the winding platform, the microfiber patch could be exposed to a different heat source (such as a laser or infrared heater) to create a fused region thereon.

Alternatively or in addition to heating the winding platform, ultrasonic welding could be used to create a fused region on the microfiber patch. The welding could be applied continuously or in select regions to create a structural pattern on the microfiber patch. Other techniques to created fused regions on the microfiber patch include compression, hot plasma, cold plasma, or chemical solvents. Yet another alternative is coating the microfiber patch with an adhesive layer of binder material after completing the windings of the microfilaments. The binder material could be a solvated polymer (such as PCL, PLA, or PDO) or other biocompatible chemicals in a suitable solvent may be used. This adhesive coating may be applied to select parts of the microfiber patch to create fused regions thereon.

Post-Winding Processing & Miscellaneous. The microfiber patch that is made on the fabrication apparatus may undergo further processing performed on or off the fabrication apparatus. For example, the method could further comprise creating openings (e.g. holes or channels) into the microfiber patch (e.g. at the fused regions). These openings could be used to facilitate instrument grasping during surgical delivery or to hold sutures. These openings could be made by any suitable technique such as hole punching, laser cutting, blade cutting, drilling, burning, or melting. Another example of further processing is making the microfiber patch relatively larger and cutting the microfiber patch into smaller individual-sized microfiber implants (i.e. batch manufacturing).

The microfiber patch made on the fabrication apparatus could be freed from the winding platform in any suitable manner. For example, the microfiber patch could be removed from the winding platform by sliding laterally on the winding platform towards one end until it is free of the winding platform. This microfiber patch may be the final product or an intermediate product that requires further processing steps to become the microfiber implant made by this method. If the microfiber patch is an intermediate product, the fabrication process would further comprise one or more additional processing steps such as hole punching, final detailing, applying coatings, laser spot welding for reinforcing, chemical treatment for microfilament cross-linking, applying an adhesive, etc. For example, the microfiber patch could be coated with a binder material that helps bind the fibers together. Examples of binders include polymer materials such as polyvinylpyrrolidone (PVP), hydroxypropyl cellulose, microcrystalline cellulose, polyethylene glycol (PEG); and biologic materials such as collagen and platelet rich plasma. Coatings of biologic materials could be lyophilized.

The final product of the fabrication process is a microfiber implant. This manufacturing process permits many variations in the design of the microfiber implant, including variations in shape, size, composition, surface smoothness/roughness, etc. This fabrication process could also create microfiber implants with complex three-dimensional geometries.

MICROFIBER IMPLANT. Making a microfiber implant in this manner imparts various unique or superior characteristics thereto that are distinguishing features. As such, another aspect of this invention is a microfiber implant having such distinguishing features. Such distinguishing features could be structural or functional. The microfiber implant could have one or more openings (e.g. holes or channels). These openings could be used to facilitate instrument grasping during surgical delivery or to hold or shuttle sutures, such as for arthroscopic delivery and fixation.

The microfiber implant comprises multiple windings of one or more microfilaments. The dimensions of the microfiber implant will vary according to the particular clinical use. For example, the microfiber implant could have a thickness in the range of 0.1-25 mm, or a length in the range of 1.0-40 cm, or a width in the range of 0.1-30 cm. The microfiber implant could have a surface area in the range of 2.0-250 cm². The microfiber implant could have one or more fused regions as described above. This microfiber implant could have 100-3,500 newtons (N) of tensile strength, which is suitable for soft tissue repairs.

The fiber density of the microfiber implant will vary according to the particular clinical use. As used herein, “fiber density” is the number of lines of microfilament (at any depth) that run across 1.0 cm span as measured in the lateral direction perpendicular to the direction of the microfilament windings (i.e. cross-cut). For example, the fiber density of the microfiber implant could be in the range of 20-750 lines of microfilament per centimeter span. As explained above, the implant could comprise multiple matting layers to increase the fiber density or implant thickness. For example, the microfiber implant could have 3-90 matting layers.

As explained above, the microfiber implant could have a coating (such as frozen and lyophilized collagen). The collagen may be from any suitable source, including human, bovine, porcine, aquatic, or any other species. The collagen could be biologically derived from organisms or made synthetically (e.g. chemical synthesis) or by recombinant technology (e.g. in cell cultures). The collagen may be full or partial length; examples of such include procollagen, telocollagen, atelocollagen, or gelatin. The collagen may further comprise any of the individual types of collagens, or multiple forms of collagen, or be mixed with other extracellular matrix components. For such coated implants, the coating could form cross-bridges between laterally adjacent strands of microfilament. The microfiber implant could have any suitable shape depending on the clinical setting for use. For example, the implant shape could be ribbon, rectangle, square, triangle, rhomboid, trapezoid, etc. The implant could be flat or have a three-dimensional shape. For example, because the implant is made by microfilament windings, the implant could have tubular shape comprising an exterior shell (of the microfilament windings) and a hollow interior void.

### BRIEF DESCRIPTION OF THE DRAWINGS

FIG. 1 shows an example fabrication apparatus of this invention.

FIG. 2 shows a close-up and partial internal view of the feeder head.

FIGS. 3A-3D show an example of how the fabrication apparatus operates. FIG. 3A shows the initial winding of the microfilament. FIG. 3B shows the result after 180° rotation of the loom frame. FIG. 3C shows the result after another 360° rotation of the loom frame. FIG. 3D shows the result after several full rotations of the loom frame.

FIGS. 4A-4E show an example of further processing of the microfiber patch. FIG. 4A shows the result after four back-and-forth windings are completed. FIG. 4B shows the result after heating the upper and lower edges of the microfiber patch. FIG. 4C shows the microfiber patch being removed off the metal rods of the loom frame. FIG. 4D shows the resulting microfiber implant. FIG. 4E shows a cross-section side view of the microfiber implant.

FIG. 5 shows another example of a loom frame that could be used in this invention.

FIG. 6 shows a flat plate mandrel as the winding platform.

FIG. 7 shows a drum mandrel as the winding platform.

FIG. 8 shows an example of how a crossbar could be used for heating.

FIG. 9 shows an example of how infrared heating could be used.

FIG. 10 shows an example of a tube-shape implant.

FIGS. 11 and 11B show an example of a block-shape implant for rotator cuff tendon repair. FIG. 11A is a perspective view; FIG. 11B is a top view.

### DETAILED DESCRIPTION OF EXAMPLE EMBODIMENTS

Drawings are provided to help understand the invention and illustrate examples of specific embodiments of the invention. The drawings herein are not necessarily made to scale or actual proportions. For example, the size of components may be adjusted to accommodate the page size.

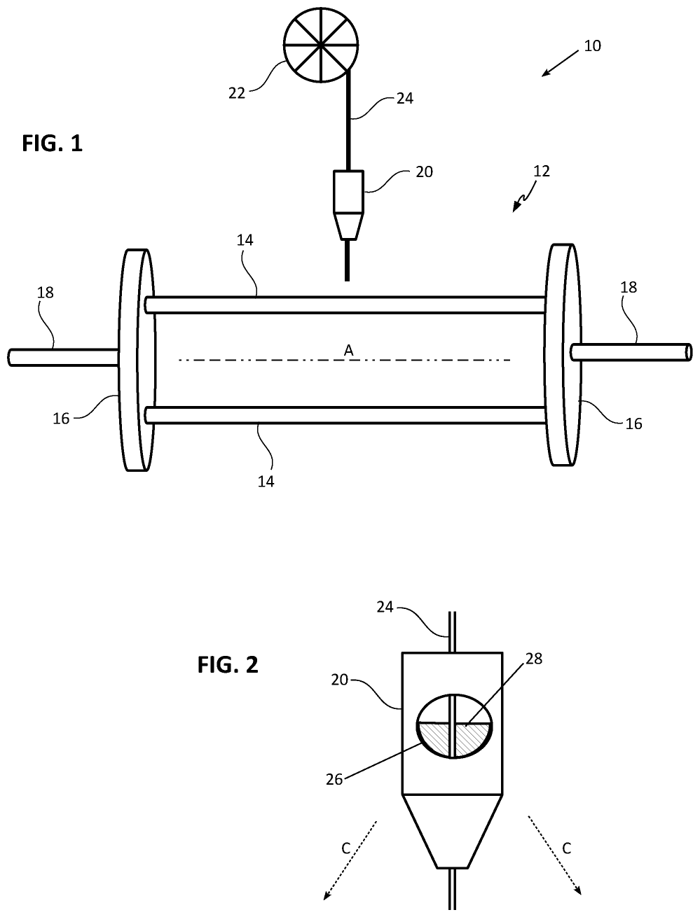

FIG. 1 shows a perspective front view of an example fabrication apparatus of this invention. Fabrication apparatus 10 uses a loom frame 12 as the rotating platform. Loom frame 12 has two cylindrical metal rods 14 upon which a microfiber implant is wound. Rods 14 are mounted on side plates 16 that hold rods 14 in parallel alignment to each other. Side plate 16 on the right side is detachable from rods 14 to facilitate removal of the microfiber patch wound thereon. Metal rods 14 have a PTFE (polytetrafluoroethylene) coating to help avoid adhering of the microfiber patch thereon. Each side plate 16 is attached to a turn shaft 18. Turn shaft 18 on the left side is spun by a motor to rotate loom frame 12 around axis A, whereas turn shaft 18 on the right side is freely rotating on a fixture.

Positioned above loom frame 12 is a stage for feeding a microfilament to the loom frame 12. The stage comprises a feeder head 20 that is mounted on a transversely oriented beam (not shown). On the transverse beam, feeder head 20 can move transversely back-and-forth relative to loom frame 12. The travel speed and angle of feeder head 20 (see below) can be varied to adjust the pitch, spacing, and layered meshing or patterning of the windings. The stage further comprises a spool 22 that stores microfilament wound thereon. Shown here is a short strand 24 of microfilament that is unwound from spool 22 and pulled into feeder head 20.

FIG. 2 shows a close-up and partial internal view of feeder head 20. Inside feeder head 20 is a coating bath 26 that contains a collagen coating solution. The coating solution contains collagen mixed into an aqueous solvent. As microfilament 24 passes through feeder head 20, it immerses in coating bath 26 and is coated with collagen before it proceeds onto loom frame 12. The orientation of feeder head 20 could be adjusted to vary the directional angle (see dashed arrows C) for passing out microfilament 24 towards loom frame 12.

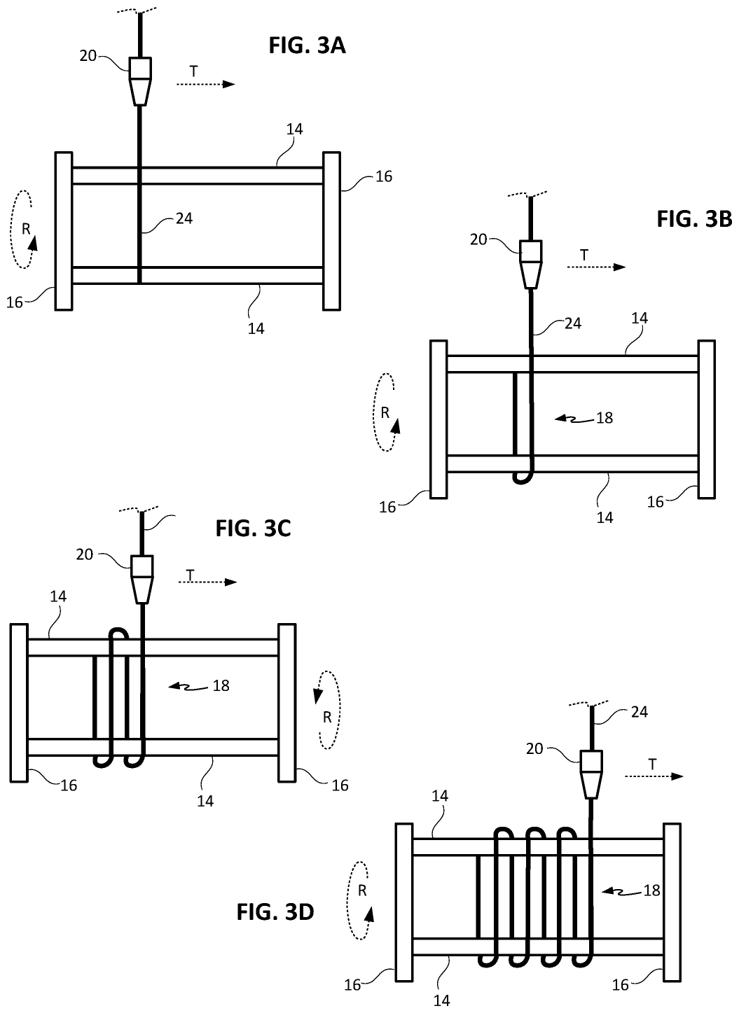

FIGS. 3A-3D show an example of how fabrication apparatus 10 operates. In FIG. 3A, the terminal end of microfilament strand 24 is adhered to bottom rod 14 (e.g. with a bioadhesive or passive winding) of loom frame 12. Microfilament strand 24 comes off spool 24 and goes into feeder head 20. Loom frame 12 rotates (see dashed arrow R) on turn shafts (not shown) under motor power. FIG. 3B shows the result after 180° rotation of loom frame 12 as feeder head 20 robotically moves transversely with high precision in the rightward direction (see dashed arrow T). This approaches one complete winding of microfilament strand 24. Note that the spacing between the windings 18 is exaggerated for better visibility.

FIG. 3C shows the result after another 360° rotation of loom frame 12 and feeder head 20 continues to travel in a rightward direction. FIG. 3D shows the result after several full rotations of loom frame 12 as feeder head 20 continues to travel in a rightward direction. The result after multiple such windings is a single layer 18 of microfiber filament. At the end of one sweep across loom frame 12, feeder head 20 reverses direction and travels towards the left for another sweep across loom frame 12. This creates another layer of microfilament windings stacked over the first winding layer 18 made in the first sweep. Feeder head 20 makes two more right and left sweeps over loom frame 12 for a total of four sweeps across loom frame 12 under computer-controlled programming.

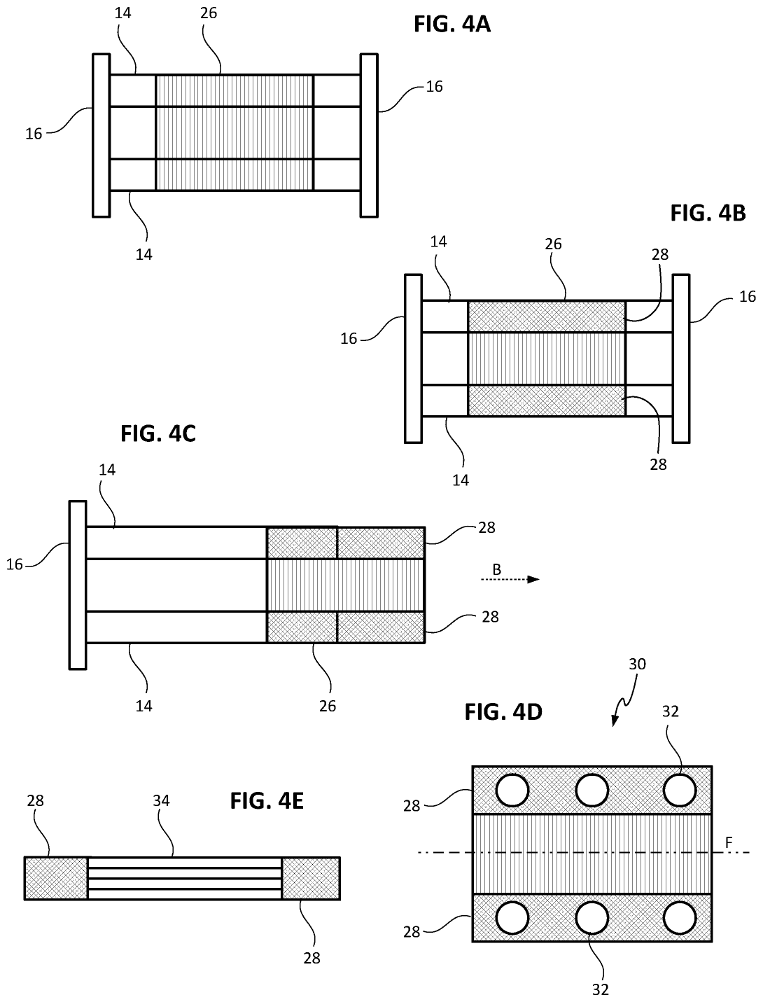

FIGS. 4A-4E show an example of further processing of microfiber patch 26. FIG. 4A shows the result after four back-and-forth windings are completed sufficient to make the desired microfiber patch 26. Rods 14 are hollow cylinders having a heating element inside. As shown in FIG. 4B, the heating element is activated and causes the upper and lower edges of microfiber patch 26 to melt and fuse. This creates two fused regions 28 at the edges of microfiber patch 26. As shown in FIG. 4C, the right side plate 16 is detached from metal rods 14 and microfiber patch 26 is slid off the right free end of metal rods 14 (see dashed arrow B). As shown in FIG. 4D, microfiber patch 26 is coated with more collagen and then holes 32 are made in fused regions 28 by laser drilling. The final result is a microfiber implant 30. Holes 32 facilitate surgical placement and fixation of microfiber implant 30. FIG. 4D also demonstrates how fiber density is measured across the direction of the windings along perpendicular axis F (in units of per centimeter span across). FIG. 4E shows a cross-section side view of microfiber implant 30. There is a stack of four layers 34 of microfilament windings.

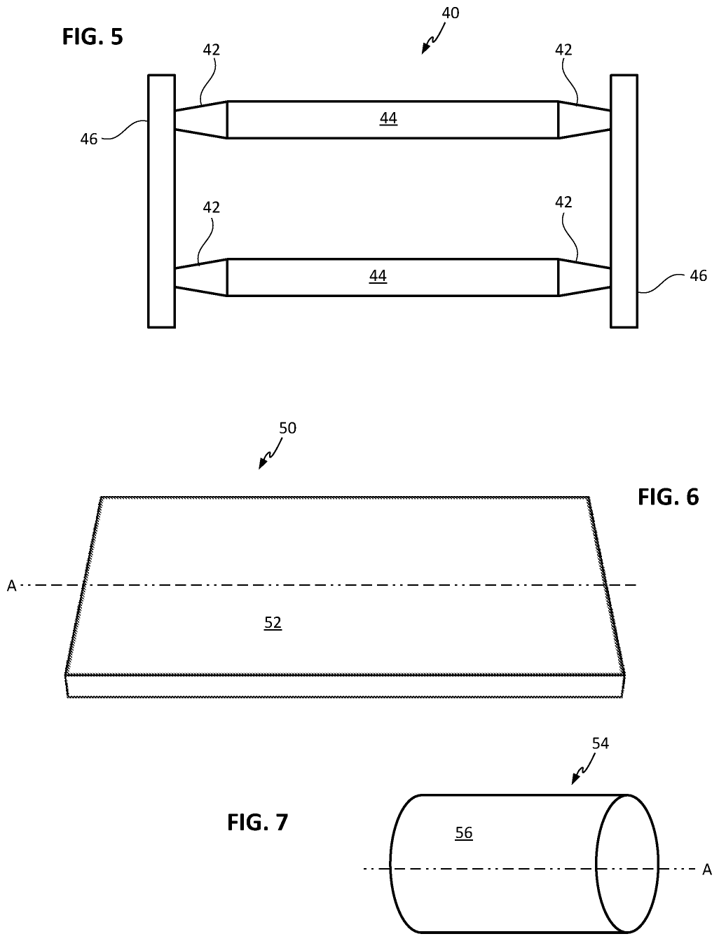

FIG. 5 shows another example of a loom frame that could be used (front view). Loom frame 40 has two cylindrical metal rods 44 upon which a microfiber implant is wound. Rods 44 are mounted on side plates 46 that hold rods 44 in parallel alignment to each other. See that rods 44 have tapered ends 42 in which the diameter gradually narrows. Having tapered ends 42 could be useful to facilitate removal of the microfiber patch wound thereon.

FIG. 6 is a perspective view showing an example of a flat plate mandrel as the winding platform. Flat plate mandrel 50 uses a flat plate 52 that rotates around transverse axis A. FIG. 7 is a perspective view showing an example of a drum mandrel as the winding platform. Drum mandrel 54 uses a hollow cylinder 56 that rotates around transverse axis A.

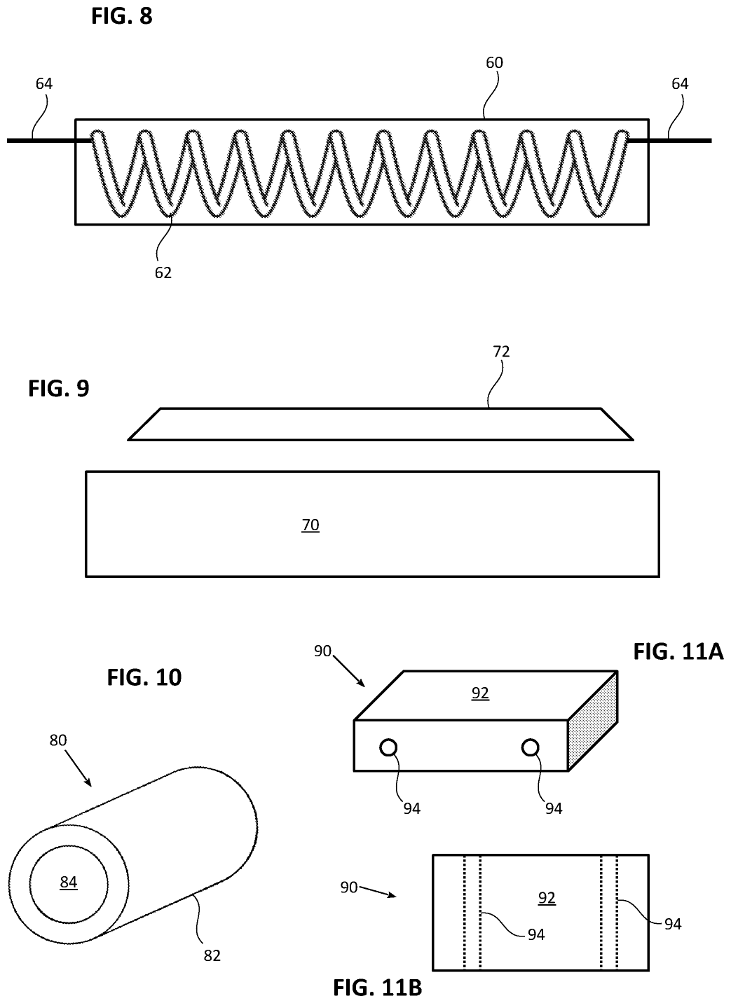

FIG. 8 shows an example of how a crossbar could be used for heating. In this internal view, hollow rod 60 (as part of a loom frame) contains a heating coil 62 inside. Heating coil 62 is connected to a power source via power supply wires 64. Electrical current is applied to heating coil 62 to heat rod 60 and form a fused region on the microfiber patch (not shown). FIG. 9 shows an example of how infrared heating could be used. Above drum mandrel 70 is an infrared heater 72. Infrared heater 72 radiates heat to the microfiber patch (not shown) on drum mandrel 70 to form a fused region on the microfiber patch.

FIG. 10 shows an example of a tube-shape implant made on a drum mandrel. Implant 80 comprises a cylindrical outer shell 82 made of windings of microfilament. Implant 80 further comprises a hollow interior void 84. Tube-shape implant 80 could be particularly useful for repairing tubular shaped body tissue such as blood vessels, nerves, respiratory tract (e.g. trachea), bones, or gastrointestinal tract (e.g. esophagus, intestines). Tube-shape implant 80 could also be useful as reinforcing sleeves for these and other body tissues (e.g. ligaments, tendons, muscle) to serve as protective and healing overlays.

FIGS. 11A (perspective view) and 11B (top view) show an example of a block-shape implant for rotator cuff tendon repair. Implant 90 comprises a rectangular block 92 made of windings of microfilament. Implant 90 further comprises two suture channels 94 that are drilled into the sides of block 92. These channels facilitate surgical (arthroscopic) delivery to the implant site. Implant 90 further has a coating of lyophilized collagen made onto block 92.

### Experimental Work

The following is a brief summary of the experimental work that was performed to validate this invention. A report with more detailed information is being submitted for journal publication. Prototype Implant Construction. Prototype microfiber implants were made using the techniques described above. The prototypes were made using poly(L-lactide) and trimethylene carbonate microfilament yarn of about 14 μm diameter size. (Non-testing samples were also made using polydioxanone and cellulose fibers to demonstrate process feasibility with other materials.) As the microfilament unwound from the spool, it was coated with collagen by passing through a collagen binder mixture in a trough. The filaments were wound on a rotating cylindrical drum mandrel. The mandrel rotation speed and feeder head movement speed were adjusted such that 1 cm width of windings were made in 97 seconds. The feeder head outputted the filament at a rate of about 143 cm/minute. The feeder head transverse travel speed was about 99 mm/minute.

Each sweep of the feeder head produced 10 windings/cm width across. A total of 11 back-and-forth sweeps were performed on the mandrel. Thus, the fiber density of the prototype implants was about 110 fibers/cm width across. The prototype implants were sized for tendon repair (2×3×0.2 mm) or ligament repair (1×3×0.2 mm). During or after the winding was completed, the prototype implants were unloaded off the mandrel and incubated at 37° C. to gel the collagen and make the implant more stable and cohesive. Further processing (as explained in detail below) was performed on the implants. For comparison, similar implants were made using conventional fused fiber fabrication (FFF) with poly(lactic acid) on a 3D printer. The process was designed to print implants with fiber lines that approximated the size and shape of the prototypes. Printing nozzle selection, speed, and height were optimized to produce the finest possible fiber lines at the tightest possible packing while avoiding fusing. The FFF implants were immersed in collagen solution to make a gelled collagen coating.

Microscopic Imaging. The prototype implants were examined by scanning electron microscopy in comparison to the conventional FFF produced implants. Fiber alignment and topology were analyzed. The prototype implants showed remarkably high fiber alignment and there were collagen resin bridges between fibers. This fiber alignment was higher compared to the FFF produced implants. On average, FFF produced fibers were over 300 μm diameter compared to about 14 μm fiber diameter in the prototype implants.

Cytocompatibility. The implants were incubated in a standard growth medium with a musculoskeletal cell type (C2C12 cells). Both the prototype and conventional FFF implants induced high metabolic activity in the cells and this was maintained through 3 days of culture. The cells also maintained healthy morphology. These results indicate that the prototype implants have high cytocompatibility.

Degradation Testing. The prototype implants were compared against the FFF produced implants for degradation over time. Testing was performed according to ASTM F1635-16 (“Standard Test Method for In Vitro Degradation Testing of Hydrolytically Degradable Polymer Resins and Fabricated Forms for Surgical Implants”). To test for mass loss from degradation, the implants were immersed in aqueous solution at 37° C. for up to 16 weeks (associated with the postoperative healing period commonly seen in soft tissue orthopedic injuries that require biomechanical support). The prototype implants exhibited a small amount of mass loss at 2 weeks duration, but not at 8 or 16 weeks duration; whereas the FFF produced implants demonstrated continuous mass loss over the entire 16 weeks duration. Both prototype and FFF implants exhibited high physical stability (retaining their shape and structure) and absence of material failure (no cracking, breaking, or thinning) through 16 weeks.

Tensile & Load/Strain Testing. Biomechanical testing was performed according to ASTM D3039M-017 (“Standard Test Method for Tensile Properties of Polymer Matrix Composite Materials”). To simulate surgical fixation, a fiber test cord was looped inside the implants for attachment to a mechanical load tester. Load was gradually increased until failure. The FFF produced implants sustained peak load (to failure) at about 12 N (newtons) initially, and this peak dropped over 30% to 7-9 N over the 16 weeks of incubation in culture media. In comparison, the prototype implants exhibited substantially superior performance. The prototype implants initially sustained 1,332 N of tensile load and retained about 1,000 N through 16 weeks of incubation in culture media. For reference, the tensile strength of the human anterior cruciate ligament (ACL) is around 1,100-1,500 N. The prototype implants also exhibited high elasticity with failure at over 70% strain, and returned to its initial shape upon cyclic loading to present a typical plastic hysteresis stress-strain curve.

Platelet Rich Plasma Wicking. Prototype implants were submerged in platelet rich plasma. The implants rapidly absorbed the plasma to about 3× their weight and continued to absorb up to 5× their weight over the 30 minute time course of testing.

Bioceramic Coating. For adhesion testing, the ends of the prototype implants were coated with carbonate apatite and β-tricalcium phosphate followed by thermal gelling at 37° C. The bioceramic coatings were retained on the implants after hydration.

Lyophilized Collagen Coating. An integrated collagen shell casing was formed around the prototype implants by immersion in collagen solution. This was then frozen and lyophilized, resulting in a collagen sponge layer over the fibers. Tensile strength of the prototype implants with the lyophilized collagen casing (under hydrated conditions) were tested against conventional electrospun collagen and polymer sheets. The results are shown in the table below. Notably, the prototype implants exhibited about 2,000 times higher suture retention strength and about 150 times higher overall strength relative to samples made by electrospun polymer and lyophilized collagen. Also, the prototypes exceeded the suture retention property of bovine Achilles tendon in direct comparison testing on the same testing machine. Also, the prototypes exceeded the suture retention strength of human supraspinatus tendon as reported in the literature. The tensile strength of the prototypes was about equal to that of native rotator cuff tendon.

| | Max Suture | | Stress at | |
|---|---|---|---|---|
| | Retention Load | Load at Failure | Failure | Youngs Modulus |
| Material Type | (N) | (N) | (MPa) | (MPa) |
| Prototypes | 1.332 ± 50.3 | 753 ± 86.2 | 14.1 ± 1.3 | 43.8 ± 11.2 |
| Electrospun Collagen- | 0.652 ± 0.043 | 4.556 ± 1.426 | 0.023 ± 0.003 | 1.0 ± 0.342 |
| Polylactide | | | | |
| Lyophilized Collagen | 1.163 ± 0.152 | 4.958 ± 2.244 | 0.098 ± 0.057 | 0.7 ± 0.122 |
| Human Rotator Cuff | 104-262 | 779.2 ± 218.9 | 21.1 ± 5.4 | 181 (M)* |
| Supraspinatus Tendon | | | | 210 (F)* |

M—Male;
F—Female;
*Interpreted from graph dataset

Conclusion. The techniques of this invention can manufacture implants with fibers similar in size and strength to native tendons and ligaments. They were three orders of magnitude stronger than similar implants made by a conventional manufacturing method.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly indicates otherwise. As such, for example, the expression “A or B” means A, or B, or both A and B. Similarly, for example, the expression “A, B, or C” means A, or B, or C, or any combination thereof.

1. A method of making a microfiber implant, comprising:
(a) having a fabrication apparatus that comprises:
a winding platform having an axis of rotation;
a feeder head that moves laterally relative to the winding platform;
(b) rotating the winding platform around the axis of rotation;
(c) feeding a microfilament into the feeder head;
(d) passing the microfilament out of the feeder head and advancing the microfilament towards the winding platform;
(e) making a winding of the microfilament on the winding platform;
(f) moving the feeder head laterally relative to the winding platform; and
(g) repeating steps (b)-(f) to make multiple windings of the microfilament on the winding platform to result in a microfiber patch;
(h) performing post-winding processing of the microfiber patch to result in the microfiber implant.

2. The method of claim 1, further comprising performing multiple sweeps of windings, wherein each sweep of windings makes a single matting layer, and the multiple sweeps results in a stack of matting layers.

3. The method of claim 2, wherein the number of sweeps is in the range of 3-50.

4. The method of claim 2, wherein each sweep makes 3-70 windings of the microfilament per centimeter across the winding platform.

5. The method of claim 1, further comprising heating the winding platform or a part thereof to create a fused region on the microfiber patch.

6. The method of claim 1, wherein the feeder head travels laterally relative to the winding platform at a speed in the range of 20-500 mm/min.

7. The method of claim 1, wherein the microfilament is passed out of the feeder head at an output rate in the range of 25-800 cm/min.

8. The method of claim 1, wherein the feeder head is capable of adjustable angle to vary a directional angle for passing out the microfilament towards the winding platform, and the method further comprises adjusting the directional angle of the feeder head while making the multiple windings.

9. The method of claim 1, wherein the post-winding processing comprises coating the microfiber patch with collagen.

10. The method of claim 9, wherein the post-winding processing further comprises freezing and lyophilizing the collagen coating.

11. The method of claim 1, wherein the post-winding processing comprises making an opening in the microfiber patch.

12. The method of claim 11, wherein the opening is a channel traveling through the microfiber patch.

13. The method of claim 1, wherein the microfilament is advanced by rotational pulling traction from rotation of the winding platform.

14. A microfiber implant made by the method of claim 1.

15. A microfiber implant comprising:
multiple windings of a microfilament, wherein the microfilament has a diameter in the range of 5-125 μm;
a coating comprising a binder material that binds the windings of microfilament together;
a channel traveling through the microfiber implant;
wherein the microfiber implant has a fiber density in the range of 20-750 lines of microfilament per centimeter span across the windings.

16. The microfiber implant of claim 15, further comprising a fused region where the windings of microfilament are fused together.

17. The microfiber implant of claim 15, having thickness in the range of 0.1-25 mm, a length in the range of 1.0-40 cm, and a width in the range of 0.1-30 cm.

18. The microfiber implant of claim 15, wherein the binder material forms cross-bridges between laterally adjacent lines of microfilament.

19. The microfiber implant of claim 15, wherein the microfiber implant has a three-dimensional shape.

20. The microfiber implant of claim 15, wherein the binder material is lyophilized collagen.
